(19) United States
(12) Patent Application Publication
(10) Pub. No.: US 2023/0111066 A1
(43) Pub. Date: Apr. 13, 2023

(54) PIGMENTED SPOT FORMATION INHIBITOR, INCREASED MELANOSOME PHAGOCYTOSIS INHIBITOR, AND EPIDERMAL DIFFERENTIATION POTENCY IMPROVER

(71) Applicant: SSP CO., LTD., Tokyo (JP)

(72) Inventor: Koichi HIYAMA, Tokyo (JP)

(21) Appl. No.: 17/938,824

(22) Filed: Oct. 7, 2022

(30) Foreign Application Priority Data
Oct. 7, 2021 (JP) 2021-165498

(51) Int. Cl. A61K 31/4415; A61P 17/00

(52) U.S. Cl. CPC A61K 31/4415; A61P 17/00

(57) ABSTRACT

A new technology for inhibiting formation of pigmented spots is provided. The technology is a pigmented spot formation inhibitor including vitamin B6 as an active ingredient.

### CROSS-REFERENCE TO RELATED APPLICATION

The instant application claims priority to Japanese Patent Application No. 2021-165498 filed on Oct. 7, 2021, the disclosure of which is incorporated herein in its entirety.

### BACKGROUND OF THE INVENTION

### Field of the Invention

The present invention relates to a pigmented spot formation inhibitor, an increased melanosome phagocytosis inhibitor, and an epidermal differentiation potency improver.

### Description of the Related Art

Pigmented spots (stains) are formed by an increase in melanin by a reduction in metabolism due to aging or UV exposure of the skin, and the formation thereof is accelerated by increased melanosome phagocytosis of epidermal keratinocytes or degradation of epidermal differentiation potency (for example, Non Patent Literature 1). Accordingly, in order to inhibit the formation of pigmented spots, it is important to inhibit an increase in melanosome phagocytosis or improve the epidermal differentiation potency.

### CITATION LIST

Non Patent Literature 1. J. Soc. Cosmet. Chem. Jpn., 51(2): 134-141
Non Patent Literature 2 Vitamin B6 Tablet 30 mg ‘F’, Pharmaceutical Interview Form, Fuji Pharma Co., Ltd., Revised April 2018 (revised 6th edition)

However, there are no reports on studies examining whether vitamin B6 typified by pyridoxine has an effect of inhibiting an increase in melanosome phagocytosis, an effect of improving epidermal differentiation potency, or an effect of inhibiting pigmented spot formation or not, although it is known that it is effective for relief of various symptoms such as angular cheilitis, cheilitis, glossitis, acute and chronic eczema, seborrheic eczema, and contact dermatitis (for example, Non Patent Literature 2).

It is an object of the present invention to provide a new technology for inhibiting formation of pigmented spots.

### SUMMARY OF THE INVENTION

The present invention can solve the problems above by the following means <1> to <13>:

<1> A pigmented spot formation inhibitor agent including vitamin B6 as an active ingredient (hereinafter, also referred to as the pigmented spot formation inhibitor of the present invention);
<2> An increased melanosome phagocytosis inhibitor agent including vitamin B6 as an active ingredient (hereinafter, also referred to as the increased melanosome phagocytosis inhibitor of the present invention);
<3> An epidermal differentiation potency improver agent including vitamin B6 as an active ingredient (hereinafter, also referred to as the epidermal differentiation potency improver of the present invention);
<4> The agent according to any one of <1> to <3>, wherein the vitamin B6 is one or more selected from the group consisting of pyridoxine, pyridoxal, pyridoxamine, and salts thereof;
<5> Use of vitamin B6 for manufacturing a pigmented spot formation inhibitor;
<6> Use of vitamin B6 for inhibiting pigmented spot formation;
<7> A method for inhibiting pigmented spot formation, comprising a step of using vitamin B6;
<8> Use of vitamin B6 for manufacturing an increased melanosome phagocytosis inhibitor;
<9> Use of vitamin B6 for inhibiting an increase in melanosome phagocytosis;
<10> A method for inhibiting an increase in melanosome phagocytosis, comprising a step of using vitamin B6;
<11> Use of vitamin B6 for manufacturing an epidermal differentiation potency improver;
<12> Use of vitamin B6 for improving epidermal differentiation potency; and
<13> A method for improving epidermal differentiation potency, comprising a step of using vitamin B6.

The increased melanosome phagocytosis inhibitor of the present invention has an excellent effect of inhibiting an increase in melanosome phagocytosis.

The epidermal differentiation potency improver of the present invention has an excellent effect of improving epidermal differentiation potency.

Accordingly, the present invention can provide a pigmented spot formation inhibitor having an effect of inhibiting pigmented spot formation.

### BRIEF DESCRIPTION OF THE DRAWINGS

The foregoing summary, as well as the following detailed description of the invention, will be better understood when read in conjunction with the appended figures. For the purpose of illustrating the invention, the figures demonstrate embodiments of the present invention. It should be understood, however, that the invention is not limited to the precise arrangements, examples, and instrumentalities shown.

FIG. 1 is a graph showing an effect of pyridoxine inhibiting an increase in melanosome phagocytosis (UVB irradiation);

FIG. 2 is a graph showing an effect of pyridoxine inhibiting an increase in melanosome phagocytosis (BSO treatment); and

FIG. 3 is a graph showing an effect of pyridoxine improving epidermal differentiation potency.

### DETAILED DESCRIPTION

The pigmented spot formation inhibitor of the present invention, the increased melanosome phagocytosis inhibitor of the present invention, and the epidermal differentiation potency improver of the present invention each include vitamin B6 as an active ingredient.

The vitamin B6 is, for example, one or more selected from the group consisting of pyridoxine, pyridoxal, pyridoxamine, and salts thereof (for example, an alkaline earth metal salt, such as a calcium salt; and an inorganic acid salt, such as hydrochloride and phosphate). Among them, from the viewpoint of the effect of inhibiting an increase in melanosome phagocytosis, effect of improving epidermal differentiation potency, and effect of inhibiting pigmented spot formation, preferred are one or more selected from pyridoxine and salts thereof, and more preferred are one or more selected from pyridoxine and pyridoxine hydrochloride.

The vitamin B6 may be a commercial product or may be synthesized by a known method.

As shown in Examples below, vitamin B6 has excellent effects of inhibiting an increase in melanosome phagocytosis and of improving epidermal differentiation potency.

Accordingly, vitamin B6 can inhibit an increase in melanosome phagocytosis of epidermal keratinocytes, can improve epidermal differentiation potency, and is useful as a pigmented spot formation inhibitor. In addition, vitamin B6 can become an increased melanosome phagocytosis inhibitor, an epidermal differentiation potency improver, or a pigmented spot formation inhibitor and can be used for inhibiting an increase in melanosome phagocytosis, improving epidermal differentiation potency, or inhibiting pigmented spot formation. Vitamin B6 can also be used for manufacturing an increased melanosome phagocytosis inhibitor, an epidermal differentiation potency improver, or a pigmented spot formation inhibitor.

The increased melanosome phagocytosis inhibitor, epidermal differentiation potency improver, and pigmented spot formation inhibitor can be used as pharmaceuticals, quasi-drugs, cosmetics, or foods effective for inhibiting an increase in melanosome phagocytosis, improving epidermal differentiation potency, or inhibiting pigmented spot formation or can be used as materials to be blended in pharmaceuticals, quasi-drugs, cosmetics, or foods.

Here, the “inhibiting an increase in melanosome phagocytosis” refers to inhibiting an increase in melanosome phagocytosis of epidermal keratinocytes, and the “improving epidermal differentiation potency” refers to improving degradation of epidermal differentiation potency (in particular, degradation of epidermal differentiation potency due to increased melanosome phagocytosis). The “inhibiting pigmented spot formation” refers to inhibiting formation of pigmented spots on the skin (in particular, formation of pigmented spots on the skin due to increased melanosome phagocytosis and/or degradation of epidermal differentiation potency).

The above-mentioned “use” can be administration or intake by a human or non-human animal and may be a therapeutic use or a non-therapeutic use. Herein, the term “non-therapeutic” refers to a concept not including medical practice, i.e., a concept not including methods for operating, treating, or diagnosing humans, more specifically, a concept not including methods for carrying out operation, treatment, or diagnosis on humans by a doctor or under the direction of a doctor.

The foods can be foods with health claims that are based on the concept of increased melanosome phagocytosis inhibition, epidermal differentiation potency improvement, or pigmented spot formation inhibition, for example, foods with functional claims, foods for specified health uses, or foods with nutrient function claims. Specific forms are, for example, tablets, capsules (soft and hard capsules), granules, drinks, and confectioneries (such as jelly, gummy candies, and chocolate) for the purpose of inhibiting an increase in melanosome phagocytosis, improving epidermal differentiation potency, inhibiting pigmented spot formation, and so on. In addition, the form can be a liquid or powder of a material to be added to a supplement or foods.

The application method of the increased melanosome phagocytosis inhibitor, epidermal differentiation potency improver, or pigmented spot formation inhibitor of the present invention may be any of oral, injection, and external skin application and is preferably oral or external skin application. Herein, the term “skin” is a concept including skin on face, body, hands, and feet and scalp.

When the increased melanosome phagocytosis inhibitor, epidermal differentiation potency improver, or pigmented spot formation inhibitor of the present invention is orally administered, examples of the dosage form include solid preparations, such as tablets, granules, and capsules; semisolid preparations; and liquid preparations. In all cases of pharmaceuticals, quasi-drugs, and foods, these dosage forms may be adopted. When used as an oral administration composition, the composition can contain, in addition to the active ingredient, additives such as an excipient, a disintegrant, a binder, and a lubricant.

When the increased melanosome phagocytosis inhibitor, epidermal differentiation potency improver, or pigmented spot formation inhibitor of the present invention is used as an external skin preparation, examples of the dosage form include an ointment, a cream, an emulsion, a gel, a paste, and a lotion. When used as these external skin preparations, the preparations can contain, in addition to the active ingredients, various ingredients that are used as base materials or additives in general external preparations. For example, solid, semi-solid, or liquid oil, such as natural animal and vegetable fat/oil, semi-synthetic fat/oil, hydrocarbon oil, higher fatty acid, higher alcohol, ester oil, silicone oil, and fluorine oil; water; alcohol, such as lower alcohol, sugar alcohol, and sterol; plant polymers, such as gum Arabic and tragacanth; microbial polymers, such as xanthan gum and dextran; starch polymers, such as carboxymethyl starch; cellulose polymers, such as carboxymethyl cellulose sodium; surfactants, such as various anionic, cationic, nonionic, and amphoteric surfactants; oil-soluble gelling agents, such as metal soap, dextrin fatty acid ester, and sucrose fatty acid ester; inorganic powder, such as titanium oxide, magnesium carbonate, mica, and hydroxyapatite; organic powder, such as polyamide powder; colored pigments; pearl pigments; moisturizing agents; preservatives; pH adjusters; chelating agents; cooling agents; anti-inflammatory agents; and skin-beautifying ingredients, such as a whitening agent, a cell activator, and a blood circulation promoter, can be used.

In the increased melanosome phagocytosis inhibitor, epidermal differentiation potency improver, and pigmented spot formation inhibitor of the present invention, the content of vitamin B6 is preferably 0.001 to 90 mass %, more preferably 0.01 to 20 mass %, and further preferably 0.1 to 10 mass %.

The increased melanosome phagocytosis inhibitor, epidermal differentiation potency improver, and pigmented spot formation inhibitor of the present invention are ingested or applied at preferably 0.1 to 100 mg, more preferably 1 to 50 mg, and further preferably 5 to 50 mg per day for an adult as vitamin B6.

### EXAMPLES

The present invention will now be described in detail with examples but is not limited to these examples.

### Test Example 1: Enhancement of Fluorescent Bead Phagocytosis by UVB Irradiation and Enhanced Phagocytosis-Inhibiting Effect by VB6

A culture medium (HuMedia-KG2: manufactured by Kurabo Industries Ltd.) was added to a 96-well plate at 0.1 mL/well, and normal human epidermal keratinocytes (NHEKs) were seeded at a density of 2.5×10⁴ cells/well, followed by incubation for 24 hours. These cells were divided into first to fourth groups. In the first group (vitamin B6+UVB group), the medium was replaced by a pyridoxine-containing culture medium (pyridoxine: 1 mM), followed by culturing for 24 hours, and irradiation with 20 mJ/cm² of UVB using a UVB broadband lamp manufactured by Koninklijke Philips N.V. was performed, followed by culturing for 24 hours. In the second group (vitamin B6 group), the medium was replaced by a pyridoxine-containing culture medium (pyridoxine: 1 mM), followed by culturing for 24 hours. In the third group (UVB group), irradiation with 20 mJ/cm² of UVB using a UVB broadband lamp manufactured by Koninklijke Philips N.V. was performed, followed by culturing for 24 hours. In the fourth group (no addition and no irradiation group), the NHEKs were directly cultured for 48 hours.

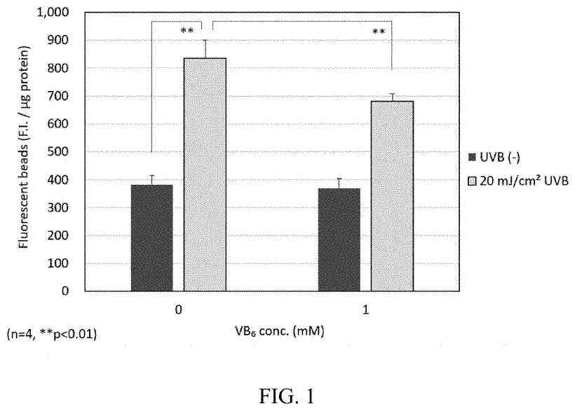

Subsequently, the NHEKs of each group were allowed to incorporate fluorescent beads (manufactured by Thermo Fisher Scientific Inc.) as pseudo-melanosomes for 4 hours, and the uptake amount of the fluorescent beads per unit protein was then measured with a microplate reader (manufactured by Tecan Group Ltd.). The results are shown in FIG. 1.

### Test Example 2: Enhancement of Fluorescent Bead Phagocytosis by BSO Treatment and Enhanced Phagocytosis-Inhibiting Effect by VB6

A culture medium (HuMedia-KG2) was added to a 96-well plate at 0.1 mL/well, and normal human epidermal keratinocytes (NHEKs) were seeded at a density of 2.5×10⁴ cells/well, followed by incubation for 24 hours. These cells were divided into first to fourth groups. In the first group (vitamin B6+BSO group), treatment with 50 μM of buthionine sulfoximine (BSO: manufactured by Sigma-Aldrich Co. LLC) was performed for 24 hours, and the medium was then replaced by a pyridoxine-containing culture medium (pyridoxine: 0.25 mM, 0.5 mM, or 1 mM), followed by culturing for 24 hours. In the second group (vitamin B6 group), the medium was replaced by a pyridoxine-containing culture medium (pyridoxine: 0.25 mM, 0.5 mM, or 1 mM), followed by culturing for 24 hours. In the third group (BSO group), treatment with 50 μM of buthionine sulfoximine (BSO) was performed for 24 hours, followed by culturing in a KG2 culture medium for 24 hours. In the fourth group (no addition group), the NHEKs were directly cultured for 48 hours.

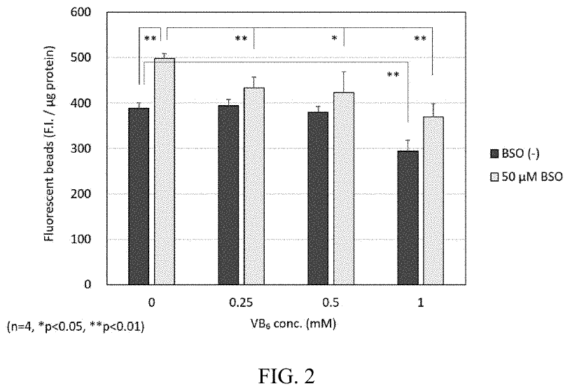

Subsequently, the NHEKs of each group were allowed to incorporate fluorescent beads (manufactured by Thermo Fisher Scientific Inc.) for 4 hours, and the uptake amount of the fluorescent beads per unit protein was then measured with a microplate reader (manufactured by Tecan Group Ltd.). The results are shown in FIG. 2.

### Test Example 3: Effect of Improving Degradation of Epidermal Differentiation Potency by Fluorescent Bead Phagocytosis

A culture medium (HuMedia-KG2) was added to a 96-well plate at 0.1 mL/well, and normal human epidermal keratinocytes (NHEKs) were seeded at a density of 2.5×10⁴ cells/well, followed by incubation for 24 hours. These cells were divided into first to third groups. In the first group (vitamin B6+bead group), the medium was replaced by a pyridoxine-containing culture medium (pyridoxine: 1 mM), followed by culturing for 24 hours. The NHEKs were allowed to incorporate fluorescent beads (manufactured by Thermo Fisher Scientific Inc.) as pseudo-melanosomes for 4 hours, followed by further culturing in a fresh culture medium (HuMedia-KG2) for 20 hours. In the second group (bead group), after culturing for 24 hours, the NHEKs were allowed to incorporate fluorescent beads (manufactured by Thermo Fisher Scientific Inc.) as pseudo-melanosomes for 4 hours, followed by further culturing in a fresh culture medium (HuMedia-KG2) for 20 hours. In the third group (control group), the NHEKs were directly cultured for 48 hours.

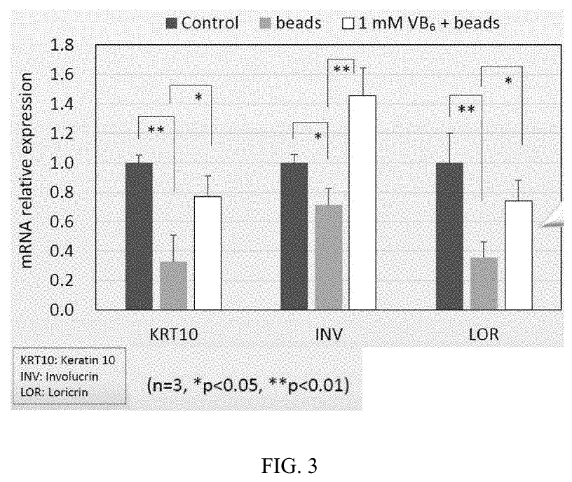

Subsequently, total RNA was extracted from the NHEKs of each group, and cDNA was produced by reverse transcription. Real-time PCR was performed using this cDNA as a template to detect gene expression levels of keratin-10, involucrin, and loricrin. The results are shown in FIG. 3.

Although the particular embodiments have been described in some detail by way of illustration and example for purposes of clarity of understanding, it is readily apparent in light of the teachings of this invention that certain changes and modifications may be made thereto without departing from the spirit or scope of the appended claims.

Accordingly, the preceding merely illustrates the principles of the invention. Various arrangements may be devised which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A pigmented spot formation inhibitor agent comprising vitamin B6 as an active ingredient.

2. The agent of claim 1, wherein the vitamin B6 is one or more selected from the group consisting of pyridoxine, pyridoxal, pyridoxamine, and salts thereof.

3. A method for inhibiting pigmented spot formation comprising administering the agent of claim 1 to a subject in need thereof.

4. An increased melanosome phagocytosis inhibitor agent comprising vitamin B6 as an active ingredient.

5. The agent of claim 4, wherein the vitamin B6 is one or more selected from the group consisting of pyridoxine, pyridoxal, pyridoxamine, and salts thereof.

6. A method of inhibiting an increase in melanosome phagocytosis comprising administering the agent of claim 4 to a subject in need thereof.

7. An epidermal differentiation potency improver agent comprising vitamin B6 as an active ingredient.

8. The agent of claim 7, wherein the vitamin B6 is one or more selected from the group consisting of pyridoxine, pyridoxal, pyridoxamine, and salts thereof.

9. A method for improving epidermal differentiation potency comprising administering the agent of claim 7 to a subject in need thereof.
